(19) United States
(12) Patent Application Publication
(10) Pub. No.: US 2021/0077539 A1
(43) Pub. Date: Mar. 18, 2021

(54) PROCESS FOR THE MANUFACTURE OF ENRICHED PHOSPHOLIPID COMPOSITIONS

(71) Applicant: ACASTI PHARMA INC., Laval (CA)

(72) Inventors: Pierre LEMIEUX, Laval (CA); Simon DESPINS, Laval (CA); Sarya AZIZ, Laval (CA); Remi LABRECQUE, Laval (CA); Nicolas BISSUEL, Laval (CA)

(21) Appl. No.: 17/054,580

(22) PCT Filed: May 15, 2019
(86) PCT No.: PCT/CA2019/050651

§ 371 Date: Nov. 11, 2020

Related U.S. Application Data
(60) Provisional application No. 62/672,180, filed on May 16, 2018.

(51) Int. Cl. A61K 35/612; B01D 3/14; B01D 11/04; B01D 21/26

(52) U.S. Cl. CPC A61K 35/612; B01D 3/143; B01D 2201/184; B01D 21/262; B01D 11/0492

(57) ABSTRACT

It is provided a process for generating enriched phospholipid compositions from raw krill oil, where such processes may be batchwise, continuous, of include both batchwise steps and continuous steps. The present technology provides high quality enriched phospholipid compositions from such methods. Moreover, the continuous process provides a significantly higher throughput compared to a batch process while concurrently providing products of equal or greater quality.

### CROSS-REFERENCE TO RELATED APPLICATIONS

The present application claims benefit of U.S. Provisional Application No. 62/672,180 filed May 16, 2018, the content of which is hereby incorporated by reference in its entirety.

### TECHNICAL FIELD

It is provided robust and flexible processes of purification for generating high quality phospholipid enriched compositions from raw krill oil for use in pharmaceutical industry.

### BACKGROUND

General methods for purification of raw krill oil have been described. U.S. 2017/0101600 and U.S. Pat. No. 9,650,590 describe a continuous process for refining of krill oil extract through a series of separation processes based on adsorption and chromatographic separation to remove salts and trimethlyamine N-oxide and recover products comprising neutral lipids, polar lipids, and astaxanthin. In US 2016/034561, a method for processing crustaceans rich in lipids to produce compositions low in fluoride, triethyl amine and trimethyl amide oxide comprising phospholipids, proteinaceous nutrients and oils through extraction with solvent is described. These methods are directed towards the removal of impurities and isolation of specific molecules. Therefore, they do not allow generation of high quality enriched phospholipid compositions for use in the pharmaceutical industry for the treatment of a medical condition such as hypertriglyceridemia.

Extraction methods for making concentrated phospholipid krill oil compositions have also been described. US 2017/0020928 discloses making concentrated phospholipid krill oil compositions which may be created using a small molecule organic solvent (i.e, acetone or ethanol)/water extraction mixture and/or sub-critical or super-critical fluid extraction at low temperatures followed by drying process. US 2016/0228461 and 2016/0228462 describe a high efficiency and high yield process for extraction of crude krill lipid compositions using alcohol fractionation. These methods allow the extraction of the lipid components from a marine biomass without any concentration. To be economically viable and in order to meet the regulatory requirements of a pharmaceutical product, the manufacturing process needs to take into consideration several variabilities (i.e, variability of the starting material etc . . . ) and should allow to produce a commercial amount of high purity product in a consistent and efficient manner.

Therefore, there remains a significant need for processes with greater overall throughput that provide commercial economically viable components and compositions derived from raw krill oil that exhibit a high purity in a consistent and efficient way for use in the pharmaceutical industry. These enriched phospholipid compositions could be used for treating hypertriglyceridemia or other indications in a subject.

### SUMMARY

It is provided a process for producing an enriched phospholipid composition comprising the steps of mixing a raw krill oil (RKO) with an organic solvent comprising at least about 85% by weight of a C₃-C₈ ketone providing a RKO-containing mixture containing an aqueous portion; and fractionating the RKO-containing mixture into a first low density layer and a first higher density phospholipid-containing layer (PCL) and separating the first PCL from the first low density layer producing the enriched phospholipid composition.

In an embodiment, the process described herein further comprises separating the first PCL from the first low density layer producing a first separated PCL; mixing the first separated PCL with the organic solvent producing a PCL containing mixture; fractionating the PCL containing mixture into a second low density layer and a second PCL; and separating the second PCL from the second low density layer producing the enriched phospholipid composition.

It is also provided a process for producing an enriched phospholipid composition comprising the steps of mixing a raw krill oil (RKO) with an organic solvent comprising at least about 85% by weight of a C₃-C₈ ketone providing a RKO-containing mixture containing an aqueous portion; fractionating the RKO-containing mixture into a first low density layer and a first higher density phospholipid-containing layer (PCL); separating the first PCL from the first low density layer producing a first separated PCL; mixing the first separated PCL with the organic solvent producing a PCL containing mixture; fractionating the PCL containing mixture into a second low density layer and a second PCL; and separating the second PCL from the second low density layer producing the enriched phospholipid composition.

In another embodiment, the process described herein further comprises filtering the RKO-containing mixture prior to the fractionating of the RKO-containing mixture.

It is further provided a process for producing an enriched phospholipid composition comprising the steps of mixing a raw krill oil (RKO) with an organic solvent comprising at least about 85% by weight of a C₃-C₈ ketone providing a RKO-containing mixture containing an aqueous portion; and mixing the RKO-containing mixture with water and fractionating the RKO-containing mixture and water into a first low density layer and a first higher density phospholipid-containing layer (PCL) and separating the first PCL from the first low density layer producing the enriched phospholipid composition.

In an additional embodiment, the process described herein further comprises combining the second PCL with a stabilizing agent, a viscosity-reducing agent, or a combination thereof.

In an embodiment, the aqueous portion is substantially free of salts.

In another embodiment, the aqueous portion is substantially free of carbonate salts, bicarbonate salts, or a combination thereof.

In another embodiment, the process encompassed herein comprises mixing at least 100 kg RKO with the organic solvent.

In a further embodiment, the process described herein comprises mixing the RKO and the organic solvent at a ratio of about 5 to about 15 in units of volume organic solvent:kg RKO.

In an embodiment, prior to mixing the RKO and the organic solvent, the process comprises heating the RKO to a temperature from about 30° C. to about 70° C.

In a further embodiment, heating the RKO is performed no more than 72 hours prior to mixing the RKO and the organic solvent.

In an additional embodiment, the mixing of raw krill oil (RKO) with the organic solvent is performed to provide the RKO-containing mixture at a temperature of about 15° C. to about 40° C.

In a further embodiment, the process further comprises directing the RKO-containing mixture through a 0.45 μm filter.

In an embodiment, the RKO-containing mixture is filtered through a first filter and a second filter.

In another embodiment, the first filter is a 10 μm filter and the second filter is a 0.45 μm filter.

In a further embodiment, the RKO-containing mixture is filtered by centrifugation or decantation.

In an embodiment, the process further comprises mixing the RKO containing mixture with water at a ratio of the RKO containing mixture versus water and the aqueous portion of about 0.15 to about 0.40 in units of volume aqueous portion:kg RKO.

In an embodiment, the first separated PCL is mixed with the organic solvent at a volume ratio of about 2 to about 10.

In another embodiment, the second PCL is combined with a viscosity-reducing agent comprising a volume ratio of viscosity-reducing agent to second PCL of about 0.1 to about 0.3.

In an embodiment, the stabilizing agent comprises Vitamin E.

In an embodiment, the stabilizing agent comprises about 3 g to about 5 g per kg of second PCL (based on dry weight of second PCL) of Vitamin E.

In a further embodiment, the process is a continuous process.

In an embodiment, the enriched phospholipid composition comprises at least 75% on a dry weight basis of phospholipids.

In an embodiment, the enriched phospholipid composition comprises at least 85% on a dry weight basis of phospholipid.

### BRIEF DESCRIPTION OF THE DRAWINGS

Reference will now be made to the accompanying drawings.

FIG. 1 illustrates a flow diagram in accordance to an embodiment of a continuous process as encompassed herein.

### DETAILED DESCRIPTION

It is provided a process for generating enriched phospholipid compositions from raw krill oil, where such processes may be batchwise, continuous, of include both batchwise steps and continuous steps.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 5% of the particular term.

A “raw krill oil” as used herein refers to oils isolated from krill, such as through methods described in U.S. Pat. Nos. 9,475,830, 9,650,590, and 9,068,142, and U.S. 2004/0234587, 2009/0074857, 2008/0274203, 2013/0274496, 2017/0020928, and 2017/0101600, the disclosures of each of which are incorporated by reference herein.

As used herein, “phospholipid” refers to an organic compound that has one fatty acid moiety attached at the sn-1 or sn-2 position of glycerol or two fatty acid moieties attached at the sn-1 and sn-2 positions of glycerol, and contains a head group linked by a phosphate residue at the sn-3 position of the glycerol. Exemplary headgroup moieties include choline, ethanolamine, serine and inositol. Phospholipids include phosphatidylcholine, lysophosphatidylcholine, phosphatidylserine, phosphatidylethanolamine, lysophosphatidylethanolamine, phosphatidylinositol, lysophosphatidylinositol, phosphatidic acid, and lysophosphatidic acid. The fatty acid moiety is the portion of the fatty acid molecule that is bound at the sn-1 or sn-2 position, for example by an ester or ether linkage. When the fatty acid moiety is a fatty acyl, the aliphatic chain of the fatty acyl is attached via an ester linkage and when the fatty acid moiety is an aliphatic chain of a fatty acid, the aliphatic chain is attached via an ether linkage. When a particular fatty acid is mentioned in connection with a phospholipid as described herein (e.g., EPA or DHA) it should therefore be taken as a reference to the relevant fatty acyl group or to its aliphatic chain.

As used herein, the term “ether phospholipid” refers to a phospholipid wherein the fatty acid moiety at one of the sn-1 or sn-2 positions is an aliphatic chain of a fatty acid attached via an ether linkage. Ether phospholipids include, for example, alkylacylphosphatidylcholine, alkylacylphosphatidylethanolamine, and alkylacylphosphatidylserine.

As used herein, the term “omega-3 fatty acid” refers to polyunsaturated fatty acids that have the double bond in the hydrocarbon chain between the third and fourth carbon atoms from the methyl end of the molecule. Non-limiting examples of omega-3 fatty acids include, 5,8,11,14,17-eicosapentaenoic acid (EPA), 4,7,10,13,16,19-docosahexaenoic acid (DHA) and 7,10,13,16,19-docosapentaenoic acid (DPA).

As used herein, the term “enriched phospholipid composition” refers to a mixture with a concentration of at least 75 wt. %, preferably 85 wt. % on a dry weight basis of phospholipids.

It is thus provided a process which includes the steps of fractionating a raw krill oil (“RKO”)-containing mixture (“the third mixture”) into a first low density layer and a first higher density phospholipid-containing layer (“PCL”) (“the first fractionation step”); separating the first PCL from the first low density layer (“the first separating step”); wherein the RKO-containing mixture comprises RKO, a first organic solvent comprising at least about 85% by weight of the solvent of a C₃-C₈ ketone, and an aqueous portion.

As encompassed herein, the process optionally further includes mixing a raw krill oil with the first organic solvent to produce a first mixture (“the first mixing step”); optionally directing the first mixture through a filter to provide a second mixture (“the first directing step”); and mixing the first mixture or the second mixture (when present) with the aqueous portion to provide the RKO-containing mixture (“the second mixing step”).

The process may include (either in addition to or in the alternative to the above optional steps) directing the first PCL to a third mixing step, the third mixing step comprising mixing the first PCL with a second organic solvent comprising at least about 85% by weight of the solvent of a C₃-C₈ ketone to provide a fourth mixture (“the second directing step”); fractionating the fourth mixture into a second low density layer and a second PCL (“the second fractionation step”); and separating the second PCL from the second low density layer (“the second separating step”).

In a related aspect, the process encompassed herein includes fractionating a raw krill oil (“RKO”)-containing mixture (“the third mixture”) into a first low density layer and a first higher density phospholipid-containing layer (“PCL”) (“the first fractionation step”); separating the first PCL from the first low density layer (“the first separating step”); directing the first PCL to a third mixing step, the third mixing step comprising mixing the first PCL with a second organic solvent comprising at least about 85% by weight of the solvent of a C₃-C₈ ketone to provide a fourth mixture (“the second directing step”); fractionating the fourth mixture into a second low density layer and a second PCL (“the second fractionation step”); and separating the second PCL from the second low density layer (“the second separating step”); wherein the RKO-containing mixture comprises RKO, a first organic solvent comprising at least about 85% by weight of the solvent of a C₃-C₈ ketone, and an aqueous portion.

The process may further include mixing a raw krill oil with the first organic solvent to produce a first mixture (“the first mixing step”); optionally directing the first mixture through a filter to provide a second mixture (“the first directing step”); and mixing the first mixture or the second mixture (when present) with the aqueous portion to provide the RKO-containing mixture (“the second mixing step”).

Thus, in any embodiment herein, the process may include mixing a raw krill oil (“RKO”) with a first organic solvent comprising at least about 85% by weight of the solvent of a C₃-C₈ ketone to produce a first mixture (“the first mixing step”); optionally directing the first mixture through a filter to provide a second mixture (“the first directing step”); mixing the first mixture or the second mixture (when present) with an aqueous portion to provide a third mixture (“the second mixing step”); fractionating the third mixture into a first low density layer and a first higher density phospholipid-containing layer (“PCL”) (“the first fractionation step”); and separating the first PCL from the first low density layer (“the first separating step”).

The process may optionally include directing the first PCL to a third mixing step, the third mixing step comprising mixing the first PCL with a second organic solvent comprising at least about 85% by weight of the solvent of a C₃-C₈ ketone to provide a fourth mixture (“the second directing step”); fractionating the fourth mixture into a second low density layer and a second PCL (“the second fractionation step”); and separating the second PCL from the second low density layer (“the second separating step”).

In any embodiment herein, the process may include mixing a raw krill oil (“RKO”) with a first organic solvent comprising at least about 85% by weight of the solvent of a C₃-C₈ ketone to produce a first mixture (“the first mixing step”); optionally directing the first mixture through a filter to provide a second mixture (“the first directing step”); mixing the second mixture with an aqueous portion to provide a third mixture (“the second mixing step”); fractionating the third mixture into a first low density layer and a first phospholipid-containing layer (“PCL”) (“the first fractionation step”); separating the first PCL from the first low density layer (“the first separating step”); directing the first PCL to a third mixing step, the third mixing step comprising mixing the first PCL with a second organic solvent comprising at least about 85% by weight of the solvent of a C₃-C₈ ketone to provide a fourth mixture (“the second directing step”); fractionating the fourth mixture into a second low density layer and a second PCL (“the second fractionation step”); and separating the second PCL from the second low density layer (“the second separating step”).

The first “low density layer” of any embodiment herein is to be understood as less dense than the first PCL (a higher density layer), and the second low density layer is less dense than the second PCL. Such low density layers and high density layers are alternately referred to herein as “light phases” and “heavy phases,” respectively, in reference to each other.

In any embodiment herein, the process may be a batchwise process, a continuous process, or a combination thereof. Accordingly, the process described herein is robust and flexible, allowing to control the variability associated with raw krill oil (i.e. season, species, age, storage and processing) which can affect its lipid content and profile and which have an important impact on the quality of the enriched phospholipid compositions. Moreover, the continuous process is a dynamic process which provides a significantly higher throughput compared to a batch process while concurrently providing products of equal or greater quality.

Thus, each step of the process may be performed as part of a batchwise process, each step of the process may be part of a continuous process, or some steps may be performed batchwise while other steps may be performed as part of a continuous process. Preferably, the first mixing step, the first directing step (when present), the second mixing step, the first fractionation step, the first separating step, the second directing step, the second fractionation step, and the second separating step are each performed as part of a continuous process. The first fractionation step may include introducing the third mixture into a horizontal settler. The first separating step may include diverting the first low density layer from the horizontal settler via a first port in the horizontal settler and diverting the first PCL from a second port in the horizontal settler. The second fractionation step may include introducing the fourth mixture into a vertical settler. The second separating step may include pumping the low density layer from the vertical settler via a first port in the vertical settler and concurrently pumping the second PCL from the vertical settler via a second port in the vertical settler.

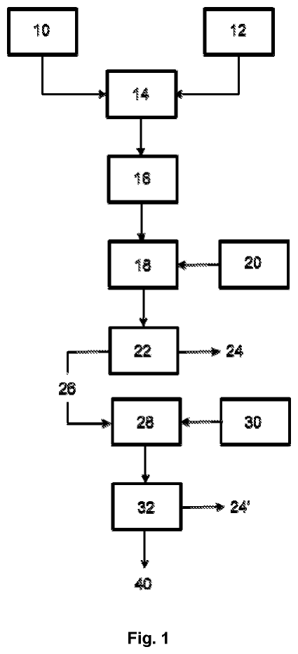

As illustrated in FIG. 1, in an embodiment, the process described herein can be a continuous process. RKO 10 and acetone 12 are first mixed 14. The resulting RKO/acetone mixture is filtered 16 using for example, but not limited to a 10 μm and 0.45 μm pore size filter.

The filtered RKO/acetone feed was directed to a static mixer 18 along with a softened water feed 20, where each feed can utilized calibrated pumps to ensure a flow rate of water and a flow rate for the filtered RKO/acetone feed. The static mixer 18 inputs to an horizontal settler 22, where a light phase 24 and a heavy phase 26 (containing phospholipids) are each continuously collected at the horizontal settler 22 extremity.

The heavy phase 26 is then directed to a second mixer 28, to which a concurrent feed of acetone 30 is also directed in an embodiment. Pumps are used to ensure the flow rate of the heavy phase 26 into the static mixer 28 and the flow rate of the acetone 30. From the mixer 28, a resulting mixture flows to a vertical settler 32 maintained providing a light phase 24′ and a heavy phospholipid-containing phase 40. Each phase is continually withdrawn from the vertical settler 32.

Addition of ethanol and Vitamin E as well as general storage conditions involving the heavy (phospholipid-containing) phase may be performed as described in the batch process or may be performed via a continuous procedure.

The first PCL and the second PCL include phospholipids. The first PCL and the second PCL may each independently include at least 75 wt. % (on a dry weight basis) phospholipids. Thus, the amount of phospholipids (on a dry weight basis) may be about 75 wt. %, about 76 wt. %, about 77 wt. %, about 78 wt. %, about 79 wt. %, about 80 wt. %, about 81 wt. %, about 82 wt. %, about 83 wt. %, about 84 wt. %, about 85 wt. %, about 86 wt. %, about 87 wt. %, about 88 wt. %, about 89 wt. %, about 90 wt. %, about 91 wt. %, about 92 wt. %, about 93 wt. %, about 94 wt. %, about 95 wt. %, or any range including and/or in between any two of these values. The first PCL and the second PCL may each independently include about 0 wt. % to about 15 wt. % free fatty acids (on a dry weight basis); thus, the amount of free fatty acids may be about 0 wt. %, about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1 wt. %, about 2 wt. %, about 3 wt. %, about 4 wt. %, about 5 wt. %, about 6 wt. %, about 7 wt. %, about 8 wt. %, about 9 wt. %, about 10 wt. %, about 11 wt. %, about 12 wt. %, about 13 wt. %, about 14 wt. %, about 15 wt. %, or any range including and/or in between any two of these values. The first PCL and the second PCL may each independently include about 0 wt. % to about 5 wt. % triglycerides (on a dry weight basis); thus, the amount of about 0 wt. %, about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1 wt. %, about 2 wt. %, about 3 wt. %, about 4 wt. %, about 5 wt. %, or any range including and/or in between any two of these values. The first PCL and the second PCL may each independently include less than about 2 wt. % monoglycerides (on a dry weight basis). The first PCL and the second PCL may each independently include monoglycerides (on a dry weight basis) of about 1.9 wt. %, about 1.8 wt. %, about 1.7 wt. %, about 1.6 wt. %, about 1.5 wt. %, about 1.4 wt. %, about 1.3 wt. %, about 1.2 wt. %, about 1.1 wt. %, about 1.0 wt. %, about 0.9 wt. %, about 0.8 wt. %, about 0.7 wt. %, about 0.6 wt. %, about 0.5 wt. %, about 0.4 wt. %, about 0.3 wt. %, about 0.2 wt. %, about 0.1 wt. %, or any range including and/or in between any two of these values, or any range lower than any one of these values. The first PCL and the second PCL may each independently include less than about 2 wt. % diglycerides (on a dry weight basis). The first PCL and the second PCL may each independently include diglycerides (on a dry weight basis) of about 1.9 wt. %, about 1.8 wt. %, about 1.7 wt. %, about 1.6 wt. %, about 1.5 wt. %, about 1.4 wt. %, about 1.3 wt. %, about 1.2 wt. %, about 1.1 wt. %, about 1.0 wt. %, about 0.9 wt. %, about 0.8 wt. %, about 0.7 wt. %, about 0.6 wt. %, about 0.5 wt. %, about 0.4 wt. %, about 0.3 wt. %, about 0.2 wt. %, about 0.1 wt. %, or any range including and/or in between any two of these values, or any range lower than any one of these values. The first PCL and the second PCL may each independently include about 0 wt. % to about 3 wt. % cholesterol (on a dry weight basis); therefore, the amount of cholesterol on a dry weight basis may be about 0 wt. %, about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1 wt. %, about 1.1 wt. %, about 1.2 wt. %, about 1.3 wt. %, about 1.4 wt. %, about 1.5 wt. %, about 1.6 wt. %, about 1.8 wt. %, about 1.9 wt. %, about 2.0 wt. %, about 2.1 wt. %, about 2.2 wt. %, about 2.3 wt. %, about 2.4 wt. %, about 2.5 wt. %, about 2.6 wt. %, about 2.8 wt. %, about 2.9 wt. %, about 3.0 wt. %, or any range including and/or in between any two of these values.

As indicated above, in any embodiment of the process may optionally include the first directing step. Such an optional step may be employed when, in performing the first mixing step, the first mixture is found to include visible insoluble components. Such insoluble components are those species that are unable to pass through a 0.45 μm filter or smaller. Filters suitable for use in the process are well appreciated by one of ordinary skill in the art, and include (but are not limited to) polyester, nylon, polypropylene, polytetrafluoroethylene, glass microfibers, or a combination of any two or more thereof. Directing the first mixture through a filter to provide a second mixture may include directing the first mixture through at least a 0.45 μm filter. Directing the first mixture through a filter to provide a second mixture may include directing the first mixture through at least a first filter and a second filter in series. By way of example, the first filter may be a 10 μm filter and the second filter may be a 0.45 μm filter. The first mixture may be passed through two or more 10 μm filters prior to passing through a 0.45 μm filter where the filters are in series. Alternatively, also encompassed are any separation methods of a solid/liquid content known in the art such as a centrifugation method or decantation.

The C₃-C₈ ketone of the first organic solvent, the second organic solvent, or both the first and second organic solvent may be acetone, butanone, 2-pentanone, 3-pentanone, methyl iso-butyl ketone, 2-hexanone, 3-hexanone, acetylacetone, or a combination of any two or more thereof. The total concentration of the C₃-C₈ ketone in the first organic solvent, the second organic solvent, or both the first and second organic solvent (expressed as wt. % of the respective organic solvent) may be about 85 wt. %, about 90 wt. %, about 95 wt. %, about 96 wt. %, about 97 wt. %, about 98 wt. %, about 99 wt. %, about 100 wt. %, or any range including and/or in between any two of these values. Thus, as a non-limiting example, in any embodiment herein the first organic solvent may be at least about 99 wt. % acetone. As another non-limiting example, the second organic solvent may be at least about 99 wt. % acetone. The first organic solvent, the second organic solvent, or both the first and second organic solvent may or may not include a co-solvent in addition to the C₃-C₈ ketone(s). Exemplary co-solvents include alcohols (e.g., methanol (CH₃OH), ethanol (EtOH), isopropanol (iPrOH), trifluoroethanol (TFE), butanol (BuOH), ethylene glycol, propylene glycol), carboxylic acids (e.g., formic acid, acetic acid, propanoic acid, butanoic acid, pentanoic acid, lauric acid, stearic acid, deoxycholic acid, glutamic acid, glucuronic acid), ethers (e.g., tetrahydrofuran (THF), 2-methyltetrahydrofuran (2Me-THF), dimethoxyethane (DME), dioxane), esters (e.g., ethyl acetate, isopropyl acetate), nitriles (e.g., acetonitrile (CH₃CN), propionitrile (CH₃CH₂CN)), or a mixture of any two or more thereof, or a mixture of any two or more thereof.

In any embodiment herein including a continuous process, the process may include mixing at least about 4 kg per hour of raw krill oil with the first organic solvent. The amount of RKO mixed per hour with the first organic solvent may be about 4 kg per hour, about 5 kg per hour, about 6 kg per hour, about 7 kg per hour, about 8 kg per hour, about 9 kg per hour, about 10 kg per hour, about 15 kg per hour, about 20 kg per hour, about 25 kg per hour, about 30 kg per hour, about 35 kg per hour, about 40 kg per hour, about 45 kg per hour, about 50 kg per hour, about 60 kg per hour, about 70 kg per hour, about 80 kg per hour, about 90 kg per hour, about 100 kg per hour, about 110 kg per hour, about 120 kg per hour, about 130 kg per hour, about 140 kg per hour, about 150 kg per hour, about 160 kg per hour, about 170 kg per hour, about 180 kg per hour, about 190 kg per hour, about 200 kg per hour, about 220 kg per hour, about 240 kg per hour, about 260 kg per hour, about 280 kg per hour, about 300 kg per hour, about 320 kg per hour, about 340 kg per hour, about 360 kg per hour, about 380 kg per hour, about 400 kg per hour, about 420 kg per hour, about 440 kg per hour, about 460 kg per hour, about 480 kg per hour, about 500 kg per hour, or any range including and/or in between any two or more of these values, or any range including and greater than any one of these values.

In any embodiment herein, the process may include mixing the raw krill oil and the first organic solvent at a ratio of about 5 to about 15 in units of volume of organic solvent:kg RKO. Thus, the ratio of volume first organic solvent to kg RKO may be about 5, about 6, about 7, about 8, about 9, about 10, about 11, about 12, about 13, about 14, about 15, or any range including and/or in between any two of these values. The mixing of the raw krill oil with the first organic solvent may be performed at a temperature of about 15° C. to about 40° C., such as about 15° C., about 16° C., about 17 ° C., about 18° C., about 19° C., about 20° C., about 22° C., about 24° C., about 26° C., about 28° C., about 30° C., about 32° C., about 34° C., about 36° C., about 38° C., about 40° C., or any range including and/or in between any two of these values. In any embodiment including a continuous process, the mixing may include combining the first organic solvent at a flow rate of about 25 L/h to about 7,500 L/h; thus, the flow rate of the first organic solvent may be about 25 L/h, about 30 L/h, about 35 L/h, about 40 L/h, about 45 L/h, about 50 L/h, about 55 L/h, about 60 L/h, about 65 L/h, about 70 L/h, about 75 L/h, about 80 L/h, about 85 L/h, about 90 L/h, about 95 L/h, about 100 L/h, about 110 L/h, about 120 L/h, about 130 L/h, about 140 L/h, about 150 L/h, about 200 L/h, about 250 L/h, about 300 L/h, about 350 L/h, about 400 L/h, about 450 L/h, about 500 L/h, about 550 L/h, about 600 L/h, about 650 L/h, about 700 L/h, about 750 L/h, about 800 L/h, about 850 L/h, about 900 L/h, about 1,000 L/h, about 1,200 L/h, about 1,400 L/h, about 1,600 L/h, about 1,800 L/h, about 2,000 L/h, about 2,200 L/h, about 2,400 L/h, about 2,600 L/h, about 2,800 L/h, about 3,000 L/h, about 3,200 L/h, about 3,400 L/h, about 3,600 L/h, about 3,800 L/h, about 4,000 L/h, about 4,200 L/h, about 4,400 L/h, about 4,600 L/h, about 4,800 L/h, about 5,000 L/h, about 5,500 L/h, about 6,000 L/h, about 6,500 L/h, about 7,000 L/h, about 7,500 L/h, or any range including and/or in between any two of these values.

Prior to mixing the RKO and first organic solvent, the process may include heating the RKO to a temperature from about 30° C. to about 70° C., such as a temperature of about 30° C., about 35° C., about 40° C., about 45° C., about 50° C., about 55° C., about 60° C., about 65° C., about 70° C., or any range including and/or in between any two of these values. In any embodiment herein, the heating of the RKO may be performed no more than 72 hours (e.g., about 24 hours to about 48 hours) prior to mixing the RKO and first organic solvent, such as no more than about 60 hours, no more than about 48 hours, no more than about 36 hours, no more than about 24 hours, no more than about 12 hours, no more than about 6 hours, no more than about 1 hour, no more than about 30 minutes, or any range including and/or in between any two of these values. In any embodiment herein, the RKO may be initially heated to one temperature as provided above and further heated en route to the mixing step to a second higher temperature as provided above, thus minimizing the duration that the RKO is at the second higher temperature.

In mixing the second mixture with an aqueous portion to provide a third mixture, the aqueous portion may be substantially free of salts. In any embodiment herein, the aqueous portion may be substantially free of a base. Exemplary bases include ammonia, basic amino acids (e.g., arginine, lysine and ornithine), carbonate salts (e.g., K₂CO₃, Na₂CO₃, (NH₄)₂CO₃), bicarbonate salts (e.g., KHCO₃, NaHCO₃), hydroxide salts (e.g., KOH, NaOH), or a combination of any two or more thereof. For example, in any embodiment herein the aqueous portion may be substantially free of carbonate salts, bicarbonate salts, or a combination thereof; in any embodiment here, the aqueous portion may be substantially free of KHCO₃ and KOH. In any embodiment herein, the aqueous portion may be substantially free of an acid. Exemplary acids include inorganic acids (such as hydrochloric acid, hydroboric acid, nitric acid, sulfuric acid, and phosphoric acid), organic acids (e.g., alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid), acidic amino acids (such as aspartic acid and glutamic acid), or a combination of any two or more thereof. In any embodiment herein, the aqueous portion may be demineralized water, deionized water, or distilled water. By way of example, deionized water exhibits a resistivity at 25° C. of at least 0.2 MΩ·cm, preferably at least about 1 MΩ·cm, and according to ASTM D1193-91 may be Type IV, III, II, or I.

As used herein, when a composition is “substantially free” of an indicated component it means that the component is present at less than 0.1 wt. % of the total referenced composition, preferably less than 0.01 wt. %.

The process may include mixing the second mixture with the aqueous portion at a ratio of about 0.10 to about 0.40 in units of volume aqueous portion to kg RKO. By kg RKO, it is to be understood this is based on kg of RKO in the first mixing step. Such a determination is made in light of the nature of the process. The volume of aqueous portion to kg RKO may be about 0.10, about 0.11, about 0.12, about 0.13, about 0.14, about 0.15, about 0.16, about 0.17, about 0.18, about 0.19, about 0.20, about 0.22, about 0.24, about 0.26, about 0.28, about 0.30, about 0.32, about 0.34, about 0.35, about 0.36, about 0.37, about 0.38, about 0.39, about 0.40, or any range including and/or in between any two of these values. The second mixing step may be performed at a temperature of about 6° C., about 8° C., about 10° C., about 12° C., about 14° C., about 16° C., about 18° C., about 20° C., about 22° C., about 24° C., about 26° C., about 28° C., about 30° C., about 32° C., about 34° C., about 36° C., about 38° C., or any range including and/or in between any two of these values.

The third mixing step of the process may include mixing the first PCL with the second organic solvent at a volume ratio first PCL: second organic solvent of about 2 to about 10; the volume ratio of the second organic solvent to the first PCL may be about 2, about 2.2, about 2.4, about 2.6, about 2.8, about 3.0, about 3.2, about 3.4, about 3.6, about 3.8, about 4.0, about 4.2, about 4.4, about 4.6, about 4.8, about 5.0, about 5.2, about 5.4, about 5.6, about 5.8, about 6.0, about 6.2, about 6.4, about 6.6, about 6.8, about 7.0, about 7.2, about 7.4, about 7.6, about 7.8, about 8.0, about 8.2, about 8.4, about 8.6, about 8.8, about 9.0, about 9.2, about 9.4, about 9.6, about 9.8, about 10.0, or any range including and/or in between any two of these values. The third mixing step may be performed at a temperature of about 15° C. to about 40° C., such as about 15° C., about 16° C., about 17° C., about 18° C., about 19° C., about 20° C., about 22° C., about 24° C., about 26° C., about 28° C., about 30° C., about 32° C., about 34° C., about 36° C., about 38° C., about 40° C., or any range including and/or in between any two of these values.

In any embodiment herein, the process may further include combining the first PCL or the second PCL with a stabilizing agent, a viscosity-reducing agent, or both, to produce a phospholipid-enriched fraction (“PLEF”). Stabilizing agents include, but are not limited to, antioxidants. Exemplary antioxidants include Vitamin A, Vitamin E, astaxanthin, canthaxanthin, β-carotene, all-trans retinol and flavonoids such as naringin, naringenin, hesperetin/kaempferol, rutin, luteolin, neohesperidin, and quecertin. In any embodiment herein, antioxidant may include Vitamin E where Vitamin E is included at about 3 g per kg of second PCL to about 5 g per kg of second PCL. The amount of Vitamin E (in grams) that may be included per kg second PCL (based on dry weight of second PCL) may be about 3, about 3.2, about 3.4, about 3.6, about 3.8, about 4.0, about 4.2, about 4.4, about 4.6, about 4.8, about 5, or any range including and/or in between any two of these values. Visocity-reducing agents include, but are not limited to, ethanol, acetone, glycerol, propylene glycol, a polyethylene glycol (e.g., PEG 300, PEG 400), arachis oil, coconut oil, castor oil, cottonseed oil, corn oil, sesame oil, soybean oil, sunflower oil, caprylic/capric triglycerides, propylene glycol diester of caprylic/capric acid, propylene glycol monolaurate, propylene glycol monocaprylate, caprylic/capric/diglyceryl succinate, medium chain fatty acid esters of propylene glycol, aerosol, cetosteryl alcohol, cetyl alcohol, flyceryl behenate, glyceryl trioctanoate, glyceryl palmitostearate, caprylic/capric/stearic triglycerides, bis-diglyceryl/caprylate/caprate/stearate/adipate, stearic acid, steryl alcohol, lauric acid, oleic acid, polyglycolized glycerides, polyoxyl-40 hydrogenated castor oil, glyceryl monocaprylate, glyceryl cocoate/citrate/lactate, glyceryl mono-di-caprylate/caprate, isosteryl diglyceryl succinate, glyceryl cocoate, glyceryl caprylate, oleoyl macrogol-8 glycerides, linoleoyl macrogolglycerides, PEG-8 caprylic/capric glycerides, propylene glycol laurate, polyglycerol dioleate, polyoxyethylene-polyoxypropylene copolymer, PEG-6 caprylic/capric glycerides, polyoxyethylene glyceryl trioleate, and polyoxyethylene(20)sorbitan monooleate, as well as those described in U.S. Pat. Publ. Nos. 2017/0182073 and 2017/0020928 (incorporated herein by reference). A volume ratio of the viscosity-reducing agent (or the total of two or more viscosity-reducing agents) to first PCL may be about 0.1 to about 0.3, such as about 0.1, about 0.12, about 0.14, about 0.16, about 0.18, about 0.20, about 0.22, about 0.24, about 0.26, about 0.28, about 0.3, or any range including and/or in between any two of these values. A volume ratio of the viscosity-reducing agent (or the total of two or more viscosity-reducing agents) to second PCL may be about 0.1 to about 0.3, such as about 0.1, about 0.12, about 0.14, about 0.16, about 0.18, about 0.20, about 0.22, about 0.24, about 0.26, about 0.28, about 0.3, or any range including and/or in between any two of these values.

In any embodiment herein wherein the process is a continuous process, the continuous process may proceed from the RKO to the second PCL and/or the PLEF at a rate of at least about 4 kg per hour of raw krill oil; thus, the rate may be about 4 kg per hour, about 5 kg per hour, about 6 kg per hour, about 7 kg per hour, about 8 kg per hour, about 9 kg per hour, about 10 kg per hour, about 15 kg per hour, about 20 kg per hour, about 25 kg per hour, about 30 kg per hour, about 35 kg per hour, about 40 kg per hour, about 45 kg per hour, about 50 kg per hour, about 60 kg per hour, about 70 kg per hour, about 80 kg per hour, about 90 kg per hour, about 100 kg per hour, about 110 kg per hour, about 120 kg per hour, about 130 kg per hour, about 140 kg per hour, about 150 kg per hour, about 160 kg per hour, about 170 kg per hour, about 180 kg per hour, about 190 kg per hour, about 200 kg per hour, about 220 kg per hour, about 240 kg per hour, about 260 kg per hour, about 280 kg per hour, about 300 kg per hour, about 320 kg per hour, about 340 kg per hour, about 360 kg per hour, about 380 kg per hour, about 400 kg per hour, about 420 kg per hour, about 440 kg per hour, about 460 kg per hour, about 480 kg per hour, about 500 kg per hour, or any range including and/or in between any two or more of these values, or any range including and greater than any one of these values.

In a related aspect a method is provided that includes substantially removing the first organic solvent, second organic solvent, and water of a PLEF produced by any embodiment herein of the process to produce a phospholipid-enriched composition. For example, such a removing step may be performed by a thin film evaporator where a thin layer of PLEF is heated under vacuum, with or without a sweeping inert gas (e.g., nitrogen). The method may further include combining one or more free fatty acids with a PLEF produced by any embodiment herein of the process, either prior to the removing step or subsequent to the removing step. The method may further include combining a mixture rich in free fatty acids with a PLEF produced by any embodiment herein of the process, either prior to the removing step or subsequent to the removing step.

In a further related aspect, a phospholipid-enriched composition produced by such any embodiment of the method is provided. The phospholipid-enriched composition may be used in treating hypertriglyceridemia in subject. The phospholipid-enriched composition may further include a pharmaceutically acceptable carrier. The term “pharmaceutically acceptable carrier” in general includes both carriers and excipients and is described further herein.

In a further related aspect, a unit-dosage form is provided that includes a phospholipid-enriched composition of any embodiment herein. The unit dosage form may be a liquid unit-dosage, included in a liquid, or included within a capsule. The capsule may comprise one or more of gelatin, a carrageenan, and hypromellose. The unit-dosage form of any embodiment herein may include an effective amount of the phospholipid-enriched composition.

“Effective amount” refers to the amount of composition required to produce a desired effect in a subject. One example of an effective amount includes amounts or dosages that yield acceptable toxicity and bioavailability levels for therapeutic (e.g., pharmaceutical) use. As used herein, a “subject” or “patient” is a mammal. Typically the subject is a human, such as a human suffering from or suspected of suffering from arthritis pain or hypertriglyceridemia. The term “subject” and “patient” can be used interchangeably.

Thus, the instant present technology provides compositions and medicaments comprising any of the enriched phospholipid compositions disclosed herein and optionally a pharmaceutically acceptable carrier or one or more excipients or fillers. The compositions may be used in the methods and treatments described herein. Such compositions and medicaments include a therapeutically effective amount of any phospholipid-enriched composition as described herein. The composition may be packaged in unit dosage form.

The examples herein are presented in order to more fully illustrate aspects of the present disclosure. The examples should in no way be construed as limiting the scope of the present disclosure.

### EXAMPLE I

### Batch Process

The following representative batch process utilized 5 kg of RKO as starting material. The RKO is weighed with a scale and transferred into a vessel already containing about 10 L of acetone under agitation. Additional acetone is then added to the vessel for a total of 50 L of acetone. The mixture is then agitated at a temperature of 30° C. Then, the RKO/acetone mixture is pumped to a container through a solvent-resistant filtration train including pre-filters of 10 μm porosity and a final filter with a porosity of 0.45 μm to provide a filtered mixture of RKO/acetone.

To the filtered mixture of RKO/acetone is added 1 L of water under constant agitation for at least 5 minutes between 20 and 25° C. The agitation is stopped and the mixture is allowed to settle in the process vessel until an upper light phase (i.e., a low density layer) and a lower heavy phase (i.e., a high density layer) are observed where the light phase is clear and a clean interphase is observed between the light and heavy phases. Upon reaching this stage, the heavy phase is transferred into another vessel and the volume calculated.

Half the volume of the heavy phase was then added under constant agitation to a vessel, followed by addition seven (7) volumes of acetone per total volume of heavy phase, and subsequently transferring the remaining half of the heavy phase to the same vessel. Agitation is continued for at least 5 minutes between 20 and 25° C. The agitation is then discontinued the resulting mixture allowed to settle in the process vessel until two phases are present (a low density light phase and a high density heavy phase), where the light phase is clear and a clean interphase is observed between the light and heavy phase. Upon achieving such settling, the heavy phase (which contains the refined phospholipids) is then transferred into a container.

The weight of the heavy phase is then determined. Absolute ethanol is added as a viscosity lowering agent in the glass vessel under constant agitation, using a ratio of 0.175 L of ethanol/L of heavy phase. A sample of the mixture is taken and analyzed to determine the dry weight of the phase. Based on its assay results, Vitamin E preparation (α-tocopherol) is manually added to the heavy phase as an antioxidant using a ratio of 4 g of Vitamin E/kg of dried heavy phase. The mixture is then agitated for at least 5 minutes between 15 and 25° C. to provide a phospholipid-enriched fraction. The total duration for the batch process from RKO to phospholipid-enriched fraction is about 24 hours or less, but can be increased if desired to provide for longer durations for phase separation and/or to account for larger-sized vessels.

For storage, the phospholipid-enriched fraction is transferred to a container under a blanket of nitrogen, hermetically sealed, and stored at 2-8° C. Average yield for the phospholipid-enriched fraction, on a dry basis (i.e., upon drying the phospholipid-enriched fraction), starting from 5 kg of RKO is 30%.

### EXAMPLE II

### Continuous Process

A continuous process was performed wherein RKO was preheated (between 30° C. and 65° C.) and acetone are fed using 2 calibrated pumps where mixing of RKO and acetone is performed using a mixing pump. For the duration of the process run, the flow rate of acetone was 56-64 L/h and the flow rate for RKO was 5.75-6.25 kg/h. The resulting RKO/acetone mixture feed was maintained at a temperature of 32.5 ±2.5° C. and filtered in-line through a 10 μm and 0.45 μm pore size filter.

The filtered RKO/acetone feed was directed to a static mixer along with a softened water feed, where each feed utilized two calibrated pumps to ensure a flow rate of water of 1.0-1.4 L/h and a flow rate for the filtered RKO/acetone feed of 62-68 L/h. The static mixer inputs to a horizontal settler, where a light phase and a heavy phase (containing phospholipids) are each continuously collected at the settler extremity. The horizontal settler is maintained at a temperature from 20-25° C.

The heavy phase is then directed to a second static mixer, to which a concurrent feed of acetone is also directed. Two calibrated pumps ensure the flow rate of the heavy phase into the static mixer is 2.2-2.6 L/h and the flow rate of the acetone is 16-18 L/h. From the static mixer a resulting mixture flows to a vertical settler maintained at a temperature of 20-25° C., where the vertical settler provides a light phase and a heavy phospholipid-containing phase. Each phase is continually withdrawn from the vertical settler.

Addition of ethanol and Vitamin E as well as general storage conditions involving the heavy (phospholipid-containing) phase may be performed as described in the batch process or may be performed via a continuous procedure. The total duration for the continuous process from RKO to phospholipid-enriched fraction is about 8 hours.

Compared to the batch process described hereinabove, the continuous process has been found to generate 3-5 times more material (of similarly high quality) with the same equipment footprint, is easier to automate, and requires less operators.

While the present disclosure has been described in connection with specific embodiments thereof, it will be understood that it is capable of further modifications and this application is intended to cover any variations, uses, or adaptations, including such departures from the present disclosure as come within known or customary practice within the art and as may be applied to the essential features hereinbefore set forth, and as follows in the scope of the appended claims.

1: A process for producing an enriched phospholipid composition comprising the steps of:
mixing a raw krill oil (RKO) with an organic solvent comprising at least about 85% by weight of a C₃-C₈ ketone providing a RKO-containing mixture containing an aqueous portion; and
fractionating the RKO-containing mixture into a first low density layer and a first higher density phospholipid-containing layer (PCL) and separating the first PCL from the first low density layer producing the enriched phospholipid composition.

2: The process of claim 1, further comprising
separating the first PCL from the first low density layer producing a first separated PCL;
mixing the first separated PCL with the organic solvent producing a PCL containing mixture;
fractionating the PCL containing mixture into a second low density layer and a second PCL; and
separating the second PCL from the second low density layer producing the enriched phospholipid composition.

3: A process for producing an enriched phospholipid composition comprising the steps of:
mixing a raw krill oil (RKO) with an organic solvent comprising at least about 85% by weight of a C₃-C₈ ketone providing a RKO-containing mixture containing an aqueous portion;
fractionating the RKO-containing mixture into a first low density layer and a first higher density phospholipid-containing layer (PCL);
separating the first PCL from the first low density layer producing a first separated PCL;
mixing the first separated PCL with the organic solvent producing a PCL containing mixture;
fractionating the PCL containing mixture into a second low density layer and a second PCL; and
separating the second PCL from the second low density layer producing the enriched phospholipid composition.

4: The process of claim 1, further comprising filtering the RKO-containing mixture prior to the fractionating of the RKO-containing mixture.

5: A process for producing an enriched phospholipid composition comprising the steps of:
mixing a raw krill oil (RKO) with an organic solvent comprising at least about 85% by weight of a C₃-C₈ ketone providing a RKO-containing mixture containing an aqueous portion; and
mixing the RKO-containing mixture with water and fractionating the RKO-containing mixture and water into a first low density layer and a first higher density phospholipid-containing layer (PCL) and separating the first PCL from the first low density layer producing the enriched phospholipid composition.

6: The process of claim 2, further comprising combining the second PCL with a stabilizing agent, a viscosity-reducing agent, or a combination thereof.

7: The process of claim 1, wherein the aqueous portion is substantially free of salts.

8: The process of claim 1, wherein the aqueous portion is substantially free of carbonate salts, bicarbonate salts, or a combination thereof.

9: The process of claim 1, wherein the process comprises mixing at least 100 kg RKO with the organic solvent.

10: The process of claim 1, wherein the process comprises mixing the RKO and the organic solvent at a ratio of about 5 to about 15 in units of volume organic solvent:kg RKO.

11: The process of claim 1, wherein prior to mixing the RKO and the organic solvent, the process comprises heating the RKO to a temperature from about 30° C. to about 70° C.

12. (canceled)

13: The process of claim 1, wherein the mixing of raw krill oil (RKO) with the organic solvent is performed to provide the RKO-containing mixture at a temperature of about 15° C. to about 40° C.

14. (canceled)

15: The process of claim 1, wherein the RKO-containing mixture is filtered through a first filter and a second filter.

16: The process of claim 1, wherein the first filter is a 10 μm filter and the second filter is a 0.45 μm filter.

17: The process of claim 1, wherein the RKO-containing mixture is filtered by centrifugation or decantation.

18: The process of claim 2, wherein the process further comprises mixing the RKO containing mixture with water at a ratio of the RKO containing mixture versus water and the aqueous portion of about 0.15 to about 0.40 in units of volume aqueous portion:kg RKO.

19: The process of claims 1, wherein the first separated PCL is mixed with the organic solvent at a volume ratio of about 2 to about 10.

20: process of claim 2, wherein the second PCL is combined with a viscosity-reducing agent comprising a volume ratio of viscosity-reducing agent to second PCL of about 0.1 to about 0.3.

21: The process of claim 2, wherein the stabilizing agent comprises Vitamin E.

22-23. (canceled)

24: The process of claim 1, wherein the enriched phospholipid composition comprises at least 75% on a dry weight basis of phospholipids.

25. (canceled)
